(19) United States
(12) Patent Application Publication
(10) Pub. No.: US 2008/0247943 A1
(43) Pub. Date: Oct. 9, 2008

(54) Blood Clot-Targeted Nanoparticles

(76) Inventors: Gregory Lanza, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(21) Appl. No.: 10/544,857

(22) PCT Filed: Aug. 20, 2003
(86) PCT No.: PCT/US03/26265

§ 371 Date: Sep. 25, 2007

Related U.S. Application Data
(63) Continuation-in-part of application No. 10/225,024, filed on Aug. 20, 2002, now Pat. No. 7,220,401.
Application No. 10/225,024 is a continuation-in-part of application No. 09/404,963, filed on Sep. 24, 1999, now Pat. No. 6,548,046.

(51) Int. Cl. A61K 51/04; A61K 49/00; A61K 9/14; A61P 7/00; A61K 49/18; A61K 49/06

(52) U.S. Cl. 424/1.37; 424/9.1; 424/490; 424/9.5; 424/9.3; 424/9.32

(57) ABSTRACT

Emulsions comprising nanoparticles formed from high boiling perfluorochemical substances, said particles coated with a lipid/surfactant coating are made target-specific by directly coupling said nanoparticles to a targeting ligand. The nanoparticle may further include biologically active agents, radionuclides, and/or other imaging agents, and are used to image and/or lyse blood clots in human subjects.

### CROSS-REFERENCE TO RELATED APPLICATIONS

This application is a continuation-in-part of U.S. Ser. No. 10/225,024 filed 20 Aug. 2002. The contents of this application are incorporated herein by reference.

### FIELD OF THE INVENTION

The invention is directed to methods to image and treat blood clots in human patients using nanoparticles which home to blood clots and that carry to these targets substances useful in diagnosis or treatment. More specifically, the invention includes the use of nanoparticles to which ligands specific for thromboses are directly bound and which further may contain imaging agents and/or bioactive materials.

### BACKGROUND ART

U.S. Pat. Nos. 5,690,907, 5,780,010 and 5,958,371, the disclosures of which are incorporated herein by reference, describe biotinylated lipid-encapsulated perfluorocarbon nanoparticles which are useful for the delivery of radionuclides, and magnetic resonance imaging agents to specific locations through a biotin-avidin system. Bioactive agents may also be included. In this approach, the target location is coupled to a target-specific ligand which is also coupled to biotin. Avidin is then employed to bridge the now biotinylated target with biotin derivatized nanoparticles contained in an emulsion. Included among the exemplified targets are blood clots; however, these blood clots are first labeled with antifibrin antibodies to which biotin is then bound. No direct targeting of blood clots with ligands specific for such clots is disclosed.

In the present invention, a ligand specific for thromboses is directly coupled, initially, to the nanoparticles in the emulsion. Thus, the emulsion, when administered, is target-specific by virtue of bearing the target-specific ligand at its surface.

Fluorochemical emulsions with specific binding moieties have been described in U.S. Pat. No. 5,401,634 for use as labels in in vitro analytical procedures. However, in vivo uses, for example, for acoustic imaging, drug delivery or delivery of imaging agents or nuclides is not contemplated. In addition, consistent with the failure to envision in vivo use, no modification of these particles for binding to thromboses is mentioned.

Others have described drug delivery using particulate supports which differ from the nanoparticles of the present invention. For example, PCT publication WO95/03829 describes oil emulsions where the drug is dispersed or solubilized inside an oil droplet and the oil droplet is targeted to a specific location by means of a ligand. U.S. Pat. No. 5,542,935 describes site-specific drug delivery using gas-filled perfluorocarbon microspheres. The drug delivery is accomplished by permitting the microspheres to home to the target and then effecting their rupture. Low boiling perfluoro compounds are used to form the particles so that the gas bubbles can form.

In contrast to the compositions described above, the compositions useful in the invention are ligand-bearing liquid emulsions based on high boiling perfluorocarbon liquids. The compositions of the invention provide facile means to deliver materials contained in their surface to blood clots.

An article reporting work of the present inventors, Flacke, S., et al., Circulation (2001) 104:1280-1285 appeared in September of 2001 and described molecular imaging of an artificially induced thrombus in canines using nanoparticles formulated with Gd-DTPA-BOA. The particles were covalently coupled to antifibrin monoclonal antibody and used to obtain magnetic resonance images of circulating blood clots. The methods described in this article, however, are not applicable to humans. The procedure described is unduly invasive, involves a double ligature, evacuation of the blood and cannulation. Further, it requires lengthy incubation with the nanoparticles in a situation of arrested blood flow.

The present invention describes procedures for imaging and treatment of human subjects using nanoparticles which target blood clots.

### DISCLOSURE OF THE INVENTION

The invention is directed to methods to image and/or effect dissolution of blood clots in humans in vivo. The invention in one aspect is directed, therefore, to methods to prepare compositions for use in such procedures. The resulting medicament/diagnostic compositions are liquid emulsions. The liquid emulsions contain nanoparticles comprised of liquid, relatively high boiling perfluorocarbons surrounded by a coating which is composed of a lipid and/or surfactant. The surrounding coating is able to couple directly to a moiety that targets blood clots or can entrap an intermediate component which is then covalently coupled to the said moiety, optionally through a linker. Alternatively, the coating may be cationic so that negatively charged blood clot targeting agents such as nucleic acids, in general or aptamers, in particular, can be adsorbed to the surface.

In addition to the targeting agent or ligand, the nanoparticles may contain at their surface a radionuclide, a contrast agent for magnetic resonance imaging (MRI) and/or a biologically active compound. The nanoparticles themselves can serve as contrast agents for ultrasound imaging or as X-ray contrast agents.

As the emulsions of the invention are intended to target blood clots or thromboses in vivo, components of clots are used as targets. Among these markers or targets are fibrin, tissue factor, gpIIb/IIIa, tissue factor/VIIA complex, activated clotting factor Xa, activated clotting factor IXa, the fibrin condensation product, d-dimer and platelets. Tissue factor is present but not preferred as it is relatively nonspecific.

Thus, in one aspect, the invention is directed to use of emulsion of liquid, high boiling perfluorocarbon-based nanoparticles, to prepare a medicament or diagnostic composition for use in in vivo methods of imaging and/or effecting the dissolution of blood clots in human subjects, and to methods to conduct said imaging or treatment. With regard to the compositions themselves, the nanoparticles further comprise a coating of a lipid/surfactant into which is embedded, or to which is directly covalently bound at least one ligand that targets blood clots, and optionally at least one biologically active compound, at least one radionuclide, and/or at least one MRI imaging agent.

The compositions prepared according to the method of the invention are useful in detecting intracardiac and intravascular thrombi. This detection is important for preventing stroke, myocardial infarction, or other sequelae of blood clotting within the circulatory system. The compositions may also contain thrombolytic agents.

### BRIEF DESCRIPTION OF THE DRAWINGS

FIGS. 1A and 1B show acoustic images obtained with fibrin-specific and non-fibrin-specific paramagnetic nanoparticles respectively. FIG. 1C shows similar images but with fat suppression.

### MODES OF CARRYING OUT THE INVENTION

The compositions of the invention are prepared for use in a method to diagnose and/or treat human subjects for conditions associated with blood clots in the circulatory system. Detection of any intracardiac and intravascular thrombus is important for prevention of stroke, myocardial infarction, and other tissue ischemia secondary to occlusive clots of arterial or venous derivation in patients presenting with appropriate symptomatology. Clots may occur in various arteries and veins, such as coronary, carotid, pulmonary, renal, subclavian and mesenteric. Examples of intracardiac clots include intraventricular mural thrombus, and atrial appendage thrombus. Intravascular thrombus includes ruptured atherosclerotic unstable plaques and other thrombus formed by vascular injury, stagnant blood flow, procoagulant states (e.g., cancer). Specific oncologic uses include detection of cancer and angiogenic beds which are associated with fibrin deposition or of other clot components.

In addition, thrombolytic or thrombus inhibitors may be incorporated onto the nanoparticle surface to dissolve any clots. Such agents include, for example, urokinase, streptokinase, tPA and the like. Incorporation of these agents onto the clot-targeted nanoparticles will generally prolong the effective drug circulatory time and increase specificity for vascular clots. Moreover, incorporation of some therapeutic agents, such as tPA, on the surface of nanoparticles will target the clot for lysis. The delivery of thrombolytic agents using compositions of the invention prevents the leakage of these lytic drugs out of the circulation into deeper sites where clots need to be retained. A major side-effect of thrombolytics given to stroke patients suffering acute myocardial infarction is cerebral and gastrointestinal hemorrhage is due to the extravasation of the lytic agents out of the vasculature and the dissolution of deep clots. The nanoparticles of the invention compositions, by virtue of their size, would be sterically hindered from reaching these sites.

In addition to thrombolytic agents, other therapeutic agents may be included in the emulsions. In addition, the nanoparticles themselves may interfere with clot formation.

As the compositions are intended to be used in human patients, relatively noninvasive methods of administration are used. The compositions would typically be introduced by intravenous injection or infusion. Other noninvasive routes are viable alternatives dependent on the application. For example, intraarterial, intralymphatic, intraperitoneal, intraurethral, intravaginal, or intracervical administration may be used. The invention compositions may also be given by local administration through catheters or direct injection into a region of the body near a target site.

In general, the aspect of the invention wherein images are obtained will be employed in non-emergency situations where the nature of the problem is unclear. Most typically, the administration of the emulsions of the invention is by an intravenous route. Typically, the dosage, measured in terms of the amount of perfluorocarbon in the nanoparticles is 0.5 g/kg or less. The amount administered of the emulsion itself is typically 0.5 cc/kg or less when the perfluorocarbon is of the order of 40% w/v. The emulsions, however, are typically diluted and infused over a time period of 10 minutes or less. However, longer time periods may be used with proper monitoring.

After with composition has been infused, images are best obtained approximately an hour after infusion, as it is estimated that it takes about two hours for all of the blood to pass through a remote site such as a coronary artery while delivery to the heart is much more rapid. As a practical matter, imaging studies are scheduled at half-hour or one-hour intervals in any event so that typical times for imaging after infusion will be 60-120 minutes, most typically 60-90 minutes after infusion.

The carrier system that is the basis for the compositions of the present invention is a nanoparticulate system containing a high boiling perfluorocarbon as a core and an outer coating that is a lipid/surfactant mixture which provides a vehicle for binding a multiplicity of copies of one or more desired components to the nanoparticle. The construction of the basic particles and the formation of emulsions containing them, regardless of the components bound to the outer surface is described in U.S. Pat. Nos. 5,690,907; 5,780,010; 5,989,520; 5,958,371 and 6,548,046 incorporated herein by reference.

The high boiling fluorochemical liquid is such that the boiling point is higher than that of body temperature—i.e., 37° C. Thus, fluorochemical liquids which have boiling points at least 30° C. are preferred, more preferably 37° C., more preferably above 50° C., and most preferably above about 90° C. The “fluorochemical liquids” useful in the invention include straight and branched chain and cyclic perfluorocarbons including perfluorinated compounds which have other functional groups. Perfluorinated compounds are preferred. Particularly preferred are compounds which will remain in the liquid state when they serve their function in the subject; for example, when used to obtain an acoustic image.

Useful perfluorocarbon emulsions are disclosed in U.S. Pat. Nos. 4,927,623, 5,077,036, 5,114,703, 5,171,755, 5,304,325, 5,350,571, 5,393,524, and 5,403,575 and include those in which the perfluorocarbon compound is perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully perfluorinated including perfluoroalkylated ether, polyether or crown ether.

The coating which comprises lipid/surfactant to form an outer coating on the nanoparticles which will contain the coupled ligand or entrap reagents for binding desired components to the surface include natural or synthetic phospholipids, fatty acids, cholesterols, lysolipids, sphingomyelins, and the like, including lipid conjugated polyethylene glycol. Various commercial anionic, cationic, and nonionic surfactants can also be employed, including Tweens, Spans, Tritons, and the like. Some surfactants are themselves fluorinated, such as perfluorinated alkanoic acids such as perfluorohexanoic and perfluorooctanoic acids, perfluorinated alkyl sulfonamide, alkylene quaternary ammonium salts and the like. In addition, perfluorinated alcohol phosphate esters can be employed. Cationic lipids included in the outer layer may be advantageous in entrapping ligands such as nucleic acids, in particular aptamers. Typical cationic lipids may include DOTMA, N-[1-(2,3-dioleoyloxy)propyl]-N,N,N-trimethylammonium chloride; DOTAP, 1,2-dioleoyloxy-3-(trimethylammonio)propane; DOTB, 1,2-dioleoyl-3-(4′-trimethyl-ammonio)butanoyl-sn-glycerol,1,2-diacyl-3-trimethylammonium-propane; 1,2-diacyl-3-dimethylammonium-propane; 1,2-diacyl-sn-glycerol-3-ethyl phosphocholine; and 3β-[N′,N′-dimethylaminoethane)-carbamol]cholesterol-HCl.

The lipid/surfactant coated nanoparticles are typically formed by microfluidizing a mixture of the fluorocarbon lipid which forms the core and the lipid/surfactant mixture which forms the outer layer in suspension in aqueous medium to form an emulsion. Sonication or other techniques may be required to obtain a suspension of the lipid/surfactant in the aqueous medium. Typically, at least one of the components of the lipid/surfactant outer layer comprises a linker or functional group which is useful to bind the targeting ligand or the targeting ligand may already be coupled to the component at the time the emulsion is prepared. The components of the outer layer may also be coupled to imaging agents or radionuclides. The components may also include biologically active materials.

For coupling by covalently binding the targeting ligand or other organic moiety (such as a chelating agent for a paramagnetic metal) to the components of the outer layer, various types of bonds and linking agents may be employed. Typical methods for forming such coupling include formation of amides with the use of carbodiamides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. A multiplicity of ways to couple, covalently, a desired ligand to one or more components of the outer layer is well known in the art. The ligand itself may be included in the surfactant layer if its properties are suitable. For example, if the ligand contains a highly lipophilic portion, it may itself be embedded in the lipid/surfactant coating. Further, if the ligand is capable of direct adsorption to the coating, this too will effect its coupling. For example, nucleic acids, because of their negative charge, adsorb directly to cationic surfactants.

By “direct binding” of the ligand to the nanoparticle is meant that the ligand specific for a component characteristic of blood clots is associated with the nanoparticle itself, as opposed to indirect binding effected through biotin/avidin. In the biotin/avidin mediated targeting methods of the art, the clot-specific ligand is coupled not to the emulsion, but rather coupled, in biotinylated form to the targeted tissue. A component “characteristic of” blood clots does not include tissue factor.

The targeting ligands cover a range of suitable moieties which bind to components of blood clots. In general, a component may itself be used to generate a ligand by using the component to raise antibodies or to select aptamers that are specific binding partners for the component. Alternatively, a suitable ligand may be known in the art. More generically, however, antibodies can be raised to desired components by conventional techniques and can be provided, preferably, as monoclonal antibodies or fragments thereof, or as single chain antibodies produced recombinantly. As the subject to be administered the compositions of the invention is human, it may be desirable to humanize antibody-type ligands using techniques generally known in the art. Further, suitable proteins or peptides which bind to targets can be discovered through phage-display techniques or through the preparation of peptide libraries using other appropriate methods. Selective aptamers which are able selectively to bind desired targets may also be prepared using known techniques such as SELEX™. (Aptamers are oligonucleotides which are selected from random pools for their ability to bind selected targets.)

In addition to the foregoing, peptidomimetics, which are small organic molecules intended to mimic peptides of known affinities can also be used as targeting agents. Particularly preferred are targeting agents that bind to fibrin, as fibrin is a particularly characteristic element included in blood clots. Antifibrin antibodies are particularly preferred, including fragments thereof, such as the F_{ab}, F_{(ab′)2} fragments, single chain antibodies (Fᵥ) and the like. In one preferred embodiment, when the emulsion includes an MRI imaging agent, such as a chelated transition metal, the targeting agent targets components of the blood clot other than fibrin, such as gpIIb/IIIa, clotting factors Xa and IXa and the like.

In addition to the ligand designed to bind the emulsion to blood clots, additional components of the emulsion can be bound to the nanoparticles in ways similar to those which are used to bind the ligands.

Other components which may be coupled to the nanoparticles through entrapment in the coating layer include radionuclides. These radionuclides include, for example, ⁹⁹Tc. The radioactive ions can be provided to the preformed emulsion in a variety of ways. For example, ⁹⁹Tc-pertecbnate may be mixed with an excess of stannous chloride and incorporated into the preformed emulsion of nanoparticles, followed by removal of unbound ⁹⁹Tc-pertechnate by repeated centrifugation and washing. Stannous oxinate can be substituted for stannous chloride. In addition, commercially available kits, such as the HM-PAO (exametazine) kit marketed as Ceretek® by Nikomed Amersham can be used. Means to attach various radioligands to the nanoparticles of the invention are understood in the art.

In addition to incorporation of radionuclides, chelating agents containing paramagnetic metals for use in magnetic resonance imaging can also be employed. Typically, a chelating agent containing a paramagnetic metal is associated with the lipids/surfactants of the coating on the nanoparticles and incorporated into the initial mixture which is sonicated. The chelating agent can be coupled directly to one or more of components of the coating layer. Suitable chelating agents include a variety of multi-dentate compounds including EDTA, DPTA, DOTA and the like. These chelating agents can be coupled directly to functional groups contained in, for example, phosphatidyl ethanolamine, bis-oleate, and the like. For use in humans, according to the present invention, DOTA is preferred. A preferred chelate is that contained in compounds of the formula:

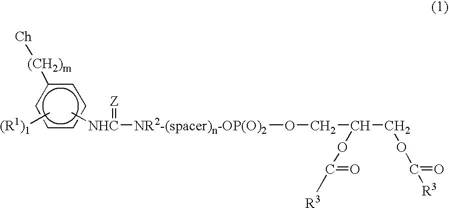

wherein Ch represents a chelating moiety;

m is 0-3;

R¹ is a non-interfering substituent;

l is 0-2;

Z is S or O;

R² is H or alkyl (1-4C);

n is 0 or 1; and

each R³ is independently an optionally substituted saturated or unsaturated hydrocarbyl group containing at least 10C, which may also comprise, associated with the chelating agent, at least one paramagnetic metal ion or a radionuclide.

The chelating agents represented by Ch typically comprise at least two, and preferably a multiplicity of nitrogens spaced by alkylene groups and to which carboxylic acid-bearing moieties are coupled. Chelating agents are characterized by comprising a multiplicity of unshared electron pairs or potential negative charges which serve to sequester the desired metal ion. Commonly employed chelating agents include porphyrins, ethylenediaminetetraacetic acid (EDTA), diethylenetriamine-N,N,N′,N″,N″-pentaacetate (DTPA), 1,4,10,13-tetraoxa-7,16-diazacyclooctadecane-7 (ODDA),16-diacetate, N-2-(azol-1(2)-yl)ethyliminodiacetic acids, 1,4,7,10-tetraazacyclododecane-N,N′,N″,N′″-tetraacetic acid (DOTA), 1,7,13-triaza-4,10,16-trioxacyclo-octadecane-N,N′,N″-triacetate (TTTA), tetraethylene glycols,1,5,9-triazacyclododecane-N,N′,N″,-tris(methylenephosphonic acid (DOTRP),N,N′,N″-trimethylammonium chloride (DOTMA) and analogues thereof. A particularly preferred chelating agent in the compounds of the invention is DOTA.

The paramagnetic metals useful in the MRI contrast agents of the invention include rare earth metals, typically, lanthanum, ytterbium, gadolinium, europium, and the like. Iron ions may also be used.

Also included in the surface of the nanoparticle, in some embodiments of the invention, are biologically active agents. These biologically active agents can be of a wide variety, including proteins, nucleic acids, pharmaceuticals, and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like. Particularly relevant are thrombolytic compounds, such as tPA, urokinase and streptokinase.

In a typical procedure for preparing the emulsions of the invention, the fluorochemical liquid and the components of the lipid/surfactant coating are fluidized in aqueous medium to form an aqueous emulsion. The functional components of the surface layer may be included in the original emulsion, or may later be covalently coupled to the surface layer subsequent to the formation of the nanoparticle emulsion. In one particular instance, for example, where a nucleic acid targeting agent or drug is to be included, the coating may employ a cationic surfactant and the nucleic acid adsorbed to the surface after the particle is formed.

When appropriately prepared, the particles contain a multiplicity of functional reagents at their outer surface, the nanoparticles typically contain thousands of molecules of MRI contrast agent. Desirably, the number of copies of a component to be coupled to the nanoparticle is in excess of 1,000 copies per particle, more preferably 5,000 copies per particle, still more preferably 10,000, and still more preferably 50,000 copies per particle.

The concentration of any biologically active agent or radionuclide will be determined by the nature of the specific agent or nuclide used. In terms of targeting agents, typically, antibody-based targeting agents are coupled to the nanoparticles at about 20-50 copies per particle. For smaller peptides and peptidomimetics or other small molecules that are used for targeting, a greater number of copies can be employed.

The particles may be prepared to include all of the auxiliary moieties in the lipid surface layer prior to emulsification, or the particles may be provided with reactive groups that are reacted with the auxiliary moieties such as MRI contrast agents, biological agents, radionuclides, and targeting agents after preparation of the emulsion. Alternatively, some of these components may be included during the preparation of the nanoparticle emulsion and others later reacted with reactive groups included in the lipid layer. If large targeting agents, such as antibodies are used, it if preferred to add them to the emulsion subsequent to preparation, since they may, by virtue of their size, interfere with the formation of the emulsion itself. A variety of ways to prepare the particles is described below.

In general, the targeted particles, directly coupled to a target-specific ligand, are useful themselves as ultrasound contrast agents. However, the inclusion of other components in multiple copies renders them useful in other respects. For instance, the inclusion of a chelating agent containing a paramagnetic ion makes the emulsion useful as a magnetic resonance imaging contrast agent. Because the particles comprise large amounts of fluorine, the addition of a paramagnetic ion is not necessary to make these particles useful for MRI. The inclusion of biologically active materials makes them useful as drug delivery systems. The inclusion of radionuclides makes them useful either as therapeutics for radiation treatment or as diagnostics for imaging or both. A multiplicity of such activities may be included; thus, images can be obtained of targeted tissues at the same time active substances are delivered to them. Finally, because the particles have a fluorocarbon core, ¹⁹F magnetic resonance imaging can be used to track the location of the particles concomitantly with their additional functions described above.

The emulsions can be prepared in a range of methods depending on the nature of the components to be included in the coating. In a typical procedure, used for illustrative purposes only, the following procedure is set forth: Perfluorooctylbromide (40% w/v, PFOB, 3M), and a surfactant co-mixture (2.0%, w/v) and glycerin (1.7%, w/v) is prepared where the surfactant co-mixture includes 64 mole % lecithin (Pharmacia Inc), 35 mole % cholesterol (Sigma Chemical Co.) and 1 mole % dipalmitoyl-L-alpha-phosphatidyl-ethanolamine, Pierce Inc.) dissolved in chloroform. A drug is suspended in methanol (˜25 μg/20 μl) and added in titrated amounts between 0.01 and 5.0 mole % of the 2% surfactant layer, preferably between 0.2 and 2.0 mole %. The chloroform-lipid mixture is evaporated under reduced pressure, dried in a 50° C. vacuum oven overnight and dispersed into water by sonication. The suspension is transferred into a blender cup (Dynamics Corporation of America) with perfluorooctylbromide in distilled or deionized water and emulsified for 30 to 60 seconds. The emulsified mixture is transferred to a Microfluidics emulsifier (Microfluidics Co.) and continuously processed at 20,000 PSI for three minutes. The completed emulsion is vialed, blanketed with nitrogen and sealed with stopper crimp seal until use. A control emulsion can be prepared identically excluding the drug from the surfactant commixture. Particle sizes are determined in triplicate at 37° C. with a laser light scattering submicron particle size analyzer (Malvern Zetasizer 4, Malvern Instruments Ltd., Southborough, Mass.), which indicate tight and highly reproducible size distribution with average diameters less than 400 nm. Unincorporated drug can be removed by dialysis or ultrafiltration techniques. To provide the targeting ligand, an F_{(ab)} fragment is coupled covalently to the phosphatidyl ethanolamine through a bifunctional linker in the procedure described above.

The following examples are intended to illustrate but not to limit the invention.

### EXAMPLE 1

### Preparation of Nanoparticles-1

Nanoparticles are prepared that comprise perfluorooctylbromide (40% w/v, PFOB), a surfactant co-mixture (2.0%, w/v) and glycerin (1.7%, w/v) and optionally an “oil” (2 to 10% w/v, substituted for the PFOB).

For various applications, the surfactant co-mixture includes therapeutic agents, dipalmitoylphosphatidyl choline, cholesterol, phosphoethanolamine-N-4 PEG(2000)-(p-maleimidophenyl)butyramide (MPB-PEG-PE) or phosphoethanolamine-(p-maleimidophenyl)butyramide, phosphatidylethanolamine, and sphingomyelin in varying molar ratios, which are dissolved in chloroform/methanol, evaporated under reduced pressure, dried in a 50° C. vacuum oven overnight and dispersed into water. For paramagnetic formulations, the surfactant co-mixture includes varying amounts of gadolinium lipophilic chelates such as gadolinium 1,4,7,10-tetraazacyclododecane-tetraacetic acid coupled to phosphatidylethanolamine through a methoxyphenyl-containing linkage (Gd-Meo-DOTA) at overall concentrations of 2.5 to 50 mole %.

Oil (i.e., vegetable oil, vitamin E or other biocompatible “oil”) may be added alone or may incorporate therapeutic agents. Lipophilic and hydrophobic therapeutic agents may be dissolved into the oil component up to supersaturating concentrations to increase total drug payload.

The above suspension is combined with PFOB and distilled, deionized water, blended and then emulsified at 10,000-20,000 PSI for three minutes.

Thiolated ligands are coupled to the maleimide derivatized phospholipid (or lipophilic substitute) in 50 mM phosphate, 10 mM EDTA buffer at pH 6.65 overnight under an nonoxidative atmosphere (i.e., nitrogen, argon). Small peptides and nonpeptide molecules are coupled to the lipid moiety prior to emulsification.

Antibodies directed to fibrin or other target contained in blood clots are reacted with N-succinimidyl S-acetylthioacetate (SATA) for 30 min, dialyzed overnight, deprotected with hydroxylamine, dialyzed in oxygen free buffers, then coupled to the nanoparticles at room temperature. Alternatively, antibodies are enzymatically digested with papain or pepsin to yield F_{(ab)} fragments isolated by routine affinity chromatography.

Particle sizes are determined in triplicate at ambient temperature with a laser light scattering submicron particle size analyzer (Malvern Zetasizer 4, Malvern Instruments Ltd, Southborough, Mass.), which typically indicates a highly reproducible size distribution with average diameters around 250 nm.

### EXAMPLE 2

### Preparation of Nanoparticles-2

In this example, a chelating ligand and a targeting ligand are coupled to the nanoparticles prior to emulsification.

The nanoparticulate emulsions in this example are comprised of 20% (w/v) fluorochemical, 2% (w/v) of a surfactant co-mixture, 1.7% (w/v) glycerin and water representing the balance. The surfactant of control, i.e., non-targeted, nanoemulsions, includes 70 mole % lecithin (Avanti Polar Lipids, Inc.), 28 mole % cholesterol (Sigma Chemical Co.), 2 mole % dipalmitoyl-phosphatidylethanolamine (DPPE) (Avanti Polar Lipids, Inc.). Fibrin-targeted nanoparticles are prepared with a surfactant co-mixture that includes: 70 mole % lecithin, 0.05 mole % N-[{w-[4-(p-maleimidophenyl)butanoyl]amino}poly(ethylene glycol)2000]1,2-distearoyl-sn-glycero-3-phosphoethanolamine (MPB-PEG-DSPE) covalently coupled to the anti-fibrin peptide such as an antibody fragment or peptidomimetic, 28 mole % cholesterol, and 1.95 mole % DPPE. The components for each nanoparticle formulation are emulsified in a M110S Microfluidics emulsifier (Microfluidics) at 20,000 PSI for four minutes. The completed emulsions are placed in crimp-sealed vials and blanketed with nitrogen. Particle sizes are determined at 37° C. with a laser light scattering submicron particle size analyzer (Malvern Instruments).

Alternatively, the DSPE-PEG (2000) maleimide mercapto acetic acid adduct,

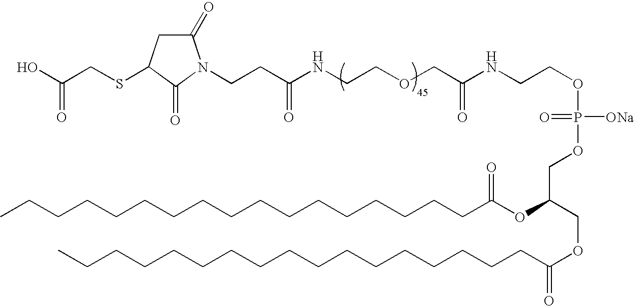

is prepared by dissolving 1,2-distearoyl-sn-glycero-3-phosphoethanolamine-N-[maleimide(polyethylene glycol)2000] in DMF and degassing by sparging with nitrogen or argon. The oxygen-free solution is adjusted to pH 7-8 using DIEA, and treated with mercaptoacetic acid. Stirring is continued at ambient temperatures until analysis indicates complete consumption of starting materials. The solution is used directly in the reaction with a peptidomimetic or small peptide. The derivatized PEG-DSPE is combined at a 1:1 molar ratio with the mimetic or small peptide in 3 ml of N₂-purged, 6 mM EDTA. The round bottom flask is then mildly sonicated in a water bath for 30 minutes under a slow stream of N₂ at 37°-40° C. The mixture is swirled occasionally to resuspend all of the lipid film. This premix is added to the remaining surfactant components, PFC and water for emulsification.

### EXAMPLE 3

### Preparation of Nanoparticles-3

In this example, the ligands for imaging and targeting are coupled to the nanoparticles after emulsification.

The nanoparticulate emulsions in this example are comprised of 20% fluorocarbon, 2% (w/v) of a surfactant co-mixture, 1.7% (w/v) glycerin and water representing the balance. The surfactant of control, i.e., non-targeted, emulsions included 70 mole % lecithin (Avanti Polar Lipids, Inc.), 28 mole % cholesterol (Sigma Chemical Co.), 2 mole % dipalmitoyl-phosphatidylethanolamine (DPPE) (Avanti Polar Lipids, Inc.). Targeted nanoparticles are prepared with a surfactant co-mixture that includes: 70 mole % lecithin, 0.05 mole % N-[{w-[4-(p-maleimidophenyl)butanoyl]amino}poly(ethylene glycol)2000]1,2-distearoyl-sn-glycero-3-phosphoethanolamine (MPB-PEG-DSPE), 28 mole % cholesterol, and 1.95 mole % DPPE. The components for each nanoparticle formulation are emulsified in a M110S Microfluidics emulsifier (Microfluidics) at 20,000 PSI for four minutes. The completed emulsions are placed in crimp-sealed vials and blanketed with nitrogen until coupled. Particle sizes are determined at 37° C. with a laser light scattering submicron particle size analyzer (Malvern Instruments).

A free thiol containing ligand (e.g., antibody, small peptide, mimetic or. antibody fragment) is dissolved in deoxygenated 50 mM sodium phosphate, 5 mM EDTA pH 6.65 buffer at a concentration of approx. 10 mg/ml. This solution is added, under nitrogen, to the nanoparticles in an equimolar ratio of the MPB-PEG₍₂₀₀₀₎-DSPE contained in the surfactant to ligand. The vial is sealed under nitrogen (or other inert gas) and allowed to react at ambient temperature with gentle agitation for a period of 4 to 16 hours. Excess (i.e., unbound) ligand may be dialyzed against phosphate/EDTA buffer using a Spectra/Por “Dispodialyzer”, 300,000 MWCO (Spectrum Laboratories, Rancho Dominguez, Calif.), if required.

For MRI imaging, the DOTA-NCS reagent of Example 5 coupled to a thiolated spacer is added.

### EXAMPLE 4

### Coupling Antibody to Fibrin to Perfluorocarbon Emulsion Particle-4

Preparation of Emulsion: The perfluorocarbon nanoparticle contrast agent is, produced by incorporating 1,2-dipalmitoyl-sn-glycero-3-phosphoethanolamine-N-4-(p-maleimidophenyl)butyramide (MPB-PE) into the outer lipid monolayer of the emulsion. The emulsion is comprised of perfluorodichlorooctane, safflower oil, a surfactant co-mixture and glycerin. The surfactant co-mixture includes lecithin, cholesterol and MPB-PE which is dissolved in chloroform. The chloroform-lipid mixture is evaporated under reduced pressure, dried in a 50° C. vacuum oven overnight and dispersed into water by sonication. The suspension is transferred into a blender cup with perfluorodichloroocatane, safflower oil and distilled, deionized water and emulsified for 30 to 60 seconds. The pre-emulsified mixture is transferred to a microemulsifier and continuously processed at 10,000 PSI for three minutes. The completed emulsion is vialed, blanketed with nitrogen and sealed with stopper crimp seal until use. A negative control emulsion is prepared identically, except a nonderivatized phosphatidylethanolamine is substituted into the surfactant co-mixture. Particle sizes are determined in triplicate at 30° C. with a laser light scatter submicron particle size analyzer.

Conjugation of fibrin F_{(ab)}′ With MPB-PE Derivatized Emulsion: F_{(ab)}′ fractions are pooled and combined with the MPB-PE derivatized emulsion (0.01 to 5.0 mg F_{(ab)}′/ml of emulsion, preferably 1 to 2 mg F_{(ab)}′/ml of emulsion). The mixture is adjusted to pH 6.7, sealed under nitrogen and allowed to react overnight at ambient temperatures with gentle, continuous mixing. The mixture may be subsequently dialyzed with a 300,000 MWCO Spectra/Por DispoDialyzer (Laguna Hills, Calif.) against 10 mM phosphate buffer (pH 7.2) to remove unconjugated F_{(ab)}′ fragments. The final emulsion is vialed under nitrogen and stored at 4° C. until use. The resulting particles contain about 50 targeting ligands per particle.

### EXAMPLE 5

### Targeted Emulsions for MRI

The emulsion is prepared as described in Example 4 but the lipid mixture includes phosphoethanolamine which has been coupled to DOTA as described below. The ratio of the coupled DOTA to the particles in the mixture is on the order of 5,000:1 or greater. The phosphatidyl ethanolamine is incorporated into the particulate surface to provide an emulsion containing nanoparticles which will then contain both antifibrin ligands and chelating agent. The chelator is then contacted with a solution of gadolinium ion to provide the finished emulsion.

Phosphoethanolamine (PE) is first coupled to t-boc protected triglycine. Standard coupling techniques, such as forming the activated ester of the free acid of the t-boc-triglycine using diisopropyl carbodiimide (or an equivalent thereof) with either N-hydroxy succinimide (NHS) or hydroxybenzotriazole (HBT) are employed and the t-boc-triglycine-PE is purified.

Treatment of the t-boc-triglycine-PE with trifluoroacetic acid yields triglycine-PE, which is then reacted with excess DOTA-NCS in DMF/CHCl₃ at 50° C. The final product is isolated by removing the solvent, followed by rinsing the remaining solid with excess water, to remove excess solvent and any un-reacted or hydrolyzed DOTA-NCS.

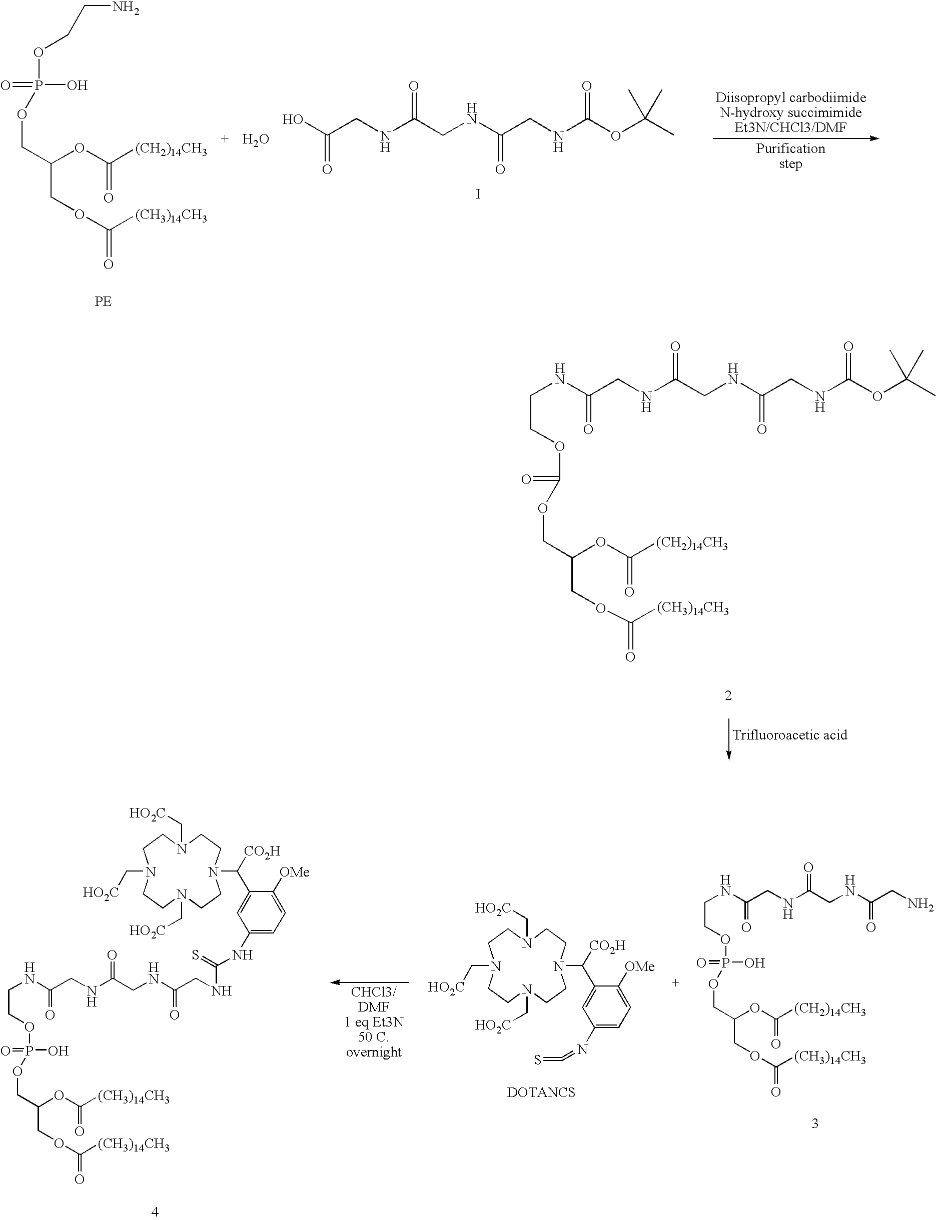

The resulting chelate coupled to PE is included in the surfactant mixture used to prepare the targeted nanoparticles of Example 4.

### EXAMPLE 6

### In Vitro Targeting of Fibrin-Rich Plasma Thrombi Using A Fibrin-Targeted, Acoustic Contrast System

Whole blood was obtained fresh and anticoagulated (9:1, v/v) with sterile sodium citrate. In a series of trials, plasma clots (9) were produced by combining plasma and 100 mM calcium chloride (3:1 v/v) with 5 units of thrombin (Sigma Chemical Company, St. Louis, Mo.) in a plastic tube overlying nitrocellulose membranes. The plasma was allowed to coagulate slowly at room temperature.

Plasma clots were incubated with anti-fibrin (F_{ab}) conjugated or non-conjugated control emulsion contrast using antifibrin monoclonal antibodies (NIB-5F3 or NIB-1H10) (Tymkewycz, et al. (1992); Tymkewycz, et al. (1993)). Half of the clots (5) were incubated individually with 150 μg biotinylated antifibrin monoclonal antibody in 10 ml PBS with 1% bovine serum albumin, (crystallized, Sigma Chemical Company, St. Louis, Mo.) for two hours; the remaining clots (4) were maintained in PBS with 1% bovine serum albumin. Bovine serum albumin was added during antibody incubations to minimize nonspecific protein binding to the polystyrene petri dish walls. The anti-fibrin targeted emulsion was incubated with clots (0.2 ml) for 30 minutes. Control clots were treated similarly with a nontargeted control perfluorocarbon emulsion (0.2 ml) for 30 minutes. The plasma clots on nitrocellulose were insonified using an acoustic microscope to assess the change in ultrasonic backscattered power attributable to the control and targeted emulsions.

The microscope consisted of a 50 MHz broadband, focused, piezoelectric delay-line transduce (¼ inch diameter, ½ inch focal length, Model V390, Panametrics Co., Waltham, Mass.) operated in the pulse-echo mode. A Tektronix DSA 601 digitizing oscilloscope (Beaverton, Oreg.) was used to digitize backscattered radiofrequency data at 500 megasamples per second with 8-bit resolution. Radiofrequency data collected from each site was averaged 32 times. Averaged radiofrequency data were acquired from approximately 400 independent sites with 50 micron lateral step resolution. The radiofrequency data are stored in a low resolution raster scan format and analyzed with custom software. Segments of the radiofrequency lines, 500 nsec in duration and encompassing surface reflection are gated for analysis. The gated data are multiplied by a Hamming window and their power spectra determined by fast-Fourier transformation.

The power spectra from each specimen was referenced to the power spectrum backscattered from a near-perfect steel plate reflector to compute the apparent frequency-dependent backscatter transfer function. The backscatter transfer function for the acoustic reflectivity of the smooth cells, B(f), was expressed in decibels relative to the power reflected from the steel plate:

B(f)²=10 log [V_{(f)}²ₜᵢₛₛᵤₑ]/[V_{(f)}²_{steel plate}]

where V_{(f)}²ₜᵢₛₛᵤₑ is the power at selected frequency of the gated rf backscattered from the cells and V_{(f)}²_{steel plate} is the power at the same frequency of the gated rf backscattered from the steel plate. Integrated backscatter (IB) was computed from the average of the frequency-dependent backscatter transfer function over the useful bandwidth of the transducer.

### EXAMPLE 7

### Targeting Canine In Situ Fibrin In Vivo

A perfluorocarbon nanoparticle contrast agent incorporates 1,2-dipalmitoyl-sn glycero-3-phosphoethanolamine-N-4-(p-maleimidophenyl)butyramide (MPB-PE; Avanti Polar Lipids, Alabaster, Ala.) into the outer lipid monolayer of the emulsion to accommodate subsequent ligand conjugation. Gd-DTPA-phosphatidylethanolamine (Gd-DTPA-PE) was added to the surfactant mixture at 0 or 20 mole % as described above.

Anti-fibrin monoclonal antibody (NIB 1H10, NIB 5F3) is produced and purified by conventional methods. A fibrin-targeted nanoparticle contrast agent is created by the covalent bonding of anti-fibrin F_{(ab)}′ fragments to the outer lipid membrane surface. Anti-fibrin F_{(ab)}′ fragments are generated (Pierce, Rockford, Ill.) and combined with the MPB-PE derivatized emulsion (1-2 mg F_{(ab)}′/ml of 40% perfluorocarbon emulsion) at pH 6.7 under nitrogen overnight. The conjugated nanoparticles are dialyzed, vialed and stored at 4° C. A nonspecific control emulsion is prepared using irrelevant IgG F_{(ab)}′ fragments.

The detection of clots in a flowing intravascular environment is evaluated in canines. Thrombi are formed within the open circulation, targeted with system in situ within isolated vascular segments, then exposed to the systemic circulation for magnetic resonance imaging. Animal protocols are approved by the Animal Studies Committee at Washington University.

Two dogs (˜20 kg) were pretreated with tranexamic acid (0.25 g/hr) to inhibit endogenous thrombolysis. Each animal was anesthetized (sodium pentothal/isoflurane), prepped for surgery and the external jugular veins exposed. Nylon monofilament (4-0) with 10, 0.5 cm strands of thrombin-soaked cotton fibers were positioned by ultrasound (Acuson Sequoia, Mountain View, Calif.). Following clot formation, thrombi were entrapped between snare closures and one ml of fibrin-targeted gadolinium or control nanoparticles was infused into the isolated segment. After contrast incubation (1 hr), the thrombi were reintroduced to the general circulation and imaged. At the conclusion of the acute procedure, animals were euthanized and the vessels retrieved for routine immunohistopathology of fibrin within the thrombus.

Canine thrombi created within the external jugular vein were imaged with a 3-D, fat-suppressed, T1-weighted fast gradient echo (TE/TR/a: 8.1/24/35f, FOV 180 mm, matrix 205×256). Flow within vessels and thrombi (as a flow deficit) were imaged with a 3-D phase contrast, T1-weighted fast gradient echo angiogram (TE/TR/a: 5.3/15/15f, FOV 200 mm, matrix 192×256).

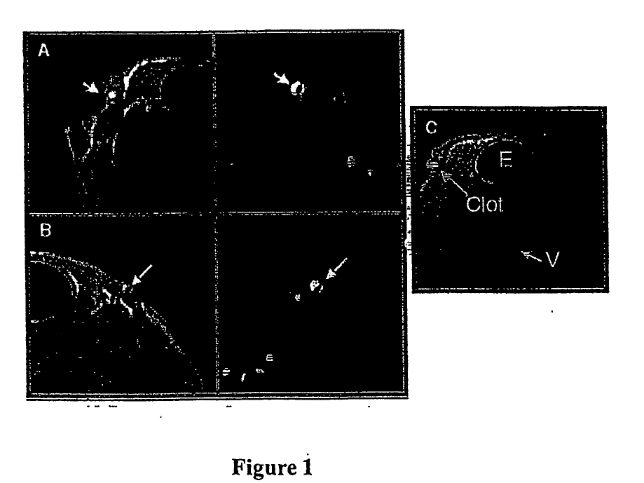

The magnitude of contrast-enhancement expected in vivo with open circulation conditions was evaluated in dogs. Control or 20 mole % (Gd-DTPA-PE) anti-fibrin nanoparticles were administered to thrombus created within the external jugular vein. Thrombus was imaged with a 3-D T1-weighted, fat suppression, fast gradient echo sequence and detectability of targeted clot was markedly enhanced by the fibrin-specific paramagnetic nanoparticles relative to control thrombus (FIGS. 1A and 1B). Phase contrast angiography revealed the clots as flow deficits in both external jugular veins. Corresponding gradient echo images revealed a selective enhancement of the treated clot yielding a signal intensity (1780±327) higher than the bright fat signal (1360±140), whereas, the control clot had a signal intensity (815±41) similar to that of the adjacent muscle (768±47). On T1-weighted gradient recalled echo images with fat suppression, the targeted clot showed the brightest image signal (FIG. 1C). The contrast-to-noise ratio (CNR) between the targeted clot and blood using nanoparticles with 20 mole % Gd-DTPA measured with this sequence was approximately 118±21. The CNR between the targeted clot and the control clot was 131±37. Fibrin immunostaining of the excised vessel and clot confirmed the abundance and localization of fibrin corresponding to the contrast enhancement in vivo.

### EXAMPLE 8

### Targeting Canine Circulating Fibrin

The perfluorocarbon nanoparticle contrast agent used in vivo (circulating) was produced by incorporating 1,2-dipalnitoyl-sn glycero-3-phosphoethanolamine-N-4-(p-maleimidophenyl)butyramide (MPB-PE; Avanti Polar Lipids, Alabaster, Ala.) into the outer lipid monolayer of the emulsion to accommodate subsequent ligand conjugation 20. Gd-DTPA-phosphatidylethanolamine (Gd-DTPA-PE) was added to the surfactant mixture at 20 mole % as described above.

Anti-fibrin monoclonal antibody (NIB 1H10, NIB 5F3) was produced and purified. A fibrin-targeted nanoparticle contrast agent was created by the covalent bonding of anti-fibrin F_{(ab)}′ fragments to the outer lipid membrane surface. Anti-fibrin F_{(ab)}′ fragments were generated (Pierce, Rockford, Ill.) and combined with the MPB-PEG-PE derivatized emulsion (1-2 mg F_{(ab)}′/ml of 40% perfluorocarbon emulsion) at pH 6.7 under nitrogen overnight. The conjugated nanoparticles were dialyzed, vialed and stored at 4° C.

Two dogs (˜20 kg) were pretreated with tranexamic acid (0.25 g/hr) to inhibit endogenous thrombolysis. Each animal was anesthetized (sodium pentothal/isoflurane), prepped for surgery and the external jugular veins exposed. Nylon monofilament (4-0) with 10, 0.5 cm strands of thrombin-soaked cotton fibers were positioned by ultrasound (Acuson Sequoia, Mountain View, Calif.). Following clot formation, thrombi were entrapped between snare closures and one ml of fibrin-targeted gadolinium or control nanoparticles was infused into the isolated segment. After contrast incubation (1 hr), the thrombi were reintroduced to the general circulation and imaged. At the conclusion of the acute procedure, animals were euthanized and the vessels retrieved for routine immunohistopathology of fibrin within the thrombus. Canine thrombi within the external jugular vein were imaged with a 3-D, fat-suppressed, T1-weighted fast gradient echo (TE/TR/a: 8.1/24/35f, FOV 180 mm, matrix 205×256). Fibrin-targeted paramagnetic nanoparticles were injected intravenously through peripheral access. After 30 minutes, T1-weighted contrast of the clot was noted. Contrast single level continued to increase up to 60 minutes.

### EXAMPLE 9

### In Vivo Human Imaging—MRI

Patient A.C., is a 35-year-old male who presents with chest tightness and shortness of breath intermittently occurring with and without modest exertion. His father died at 40 years of age from a sudden heart attack. A.C. visits his doctor and undergoes an EKG, echocardiogram and a treadmill stress test. All are unremarkable. Given his past history, his doctor elects a noninvasive MRI study of his heart. The patient's cardiac function is normal and an MRI angiogram suggests mild diffuse coronary disease without a focal stenosis.

The patient is given fibrin-targeted nanoparticles as described in Example 2, comprising a F_{(ab)}′ region of antifibrin antibodies and further modified to incorporate chelated gadolinium as described in PCT publication PCT/US03/09277, incorporated herein by reference, but substituting DOTA for DPTA as the chelator.

The emulsion is infused intravenously at a dosage of 0.5 cc/kg over 10 minutes. The patient waits about one hour in the waiting room and then returns to the MRI imaging area. MRI images of coronary arteries and heart reveal a series of tightly clustered ruptures of the mid right coronary artery. The patient is placed on medical antithrombotic therapy and is transferred to a cardiac catheterization lab where he undergoes stent placement at the specific site of antifibrin nanoparticle contrast to reinforce the rupturing vascular wall, preventing a more serious breach of the vascular wall with ensuing coronary occlusion and myocardial infarction.

### EXAMPLE 10

### In Vivo Human Imaging—Acoustic

Patient B.L., is a 65-year-old male with known hypercholesterolemia, hypertension and 30 pack-year history of smoking. B.L. awakes one morning and notes numbness and weakness in his left leg which gradually resolves over the next two hours. Concerned, B.L. visits his doctor who performs a brief physical exam which is within normal limits. The patient remains concerned and the doctor agrees to order a duplex ultrasound study of his carotid arteries to rule out high grade vascular occlusion. The study reveals no hemodynamically significant stenoses. While in the ultrasound lab, a decision is made to administer fibrin-targeted nanoparticles, comprised of the variable region of an antifibrin antibody coupled to the surface of the acoustically reflective nanoparticle as described in Example 4. The agent is administered by intravenous infusion at a dosage of 0.5 cc/kg over 10 minutes and the patient is reexamined with 2D and 3D ultrasound.

### EXAMPLE 11

### Administration of Thrombolytic Drugs

Patient D.S., is a 40-year-old male presenting with symptoms of acute myocardial infarction diagnosed by history, exam and EKG at a rural hospital without interventional cardiology capability. The patient suggests he may have had a transient ischemic attack about a year previous and his blood pressure is currently 180/110. The patient is given fibrin-targeted nanoparticles prepared as in Example 4 which bear recombinant tissue plasminogen activator on their surface to minimize the risk of intracranial bleeding potential. The infusion is given over 10 minutes, intravenously at a dosage of 0.5 cc/kg bearing 100 mg of rTPA. The patient's chest discomfort subsides to near normal in 10 minutes and he is sent to a tertiary medical care center by air rescue in stable condition for further cardiovascular evaluation.

### EXAMPLE 12

Mr. G., is a 60-year-old male who has been in good general health but has noted two instances of chest heaviness lasting five minutes while working in the yard over the last two months. Both episodes were associated with mild light headedness and increased diaphoresis. The patient indicates that he suspected he was over exerting himself in the “hot” weather and simply needed to take a short rest in the shade. Today, Mr. G. noted a similar but brief episode of chest discomfort while racing to catch a train to work. He calls his doctor. He says he knows he needs to loose some weight a stop smoking but could something else be going on. The doctor suggests an exercise stress test.

Mr. G. undergoes a nuclear exercise stress test. His tolerance to exercise is only fair, the study is read as normal. Given the clinical history, the doctor believes the patient may have significant coronary artery disease. The doctor feels the evidence does not warrant invasive cardiac testing and questions the likelihood of detecting focal high grade stenosis. He submits the patient for a noninvasive MR angiogram which confirms mild diffuse coronary disease with out high grade stenosis. Mr. G. is given by IV infusion fibrin-targeted paramagnetic nanoparticles as prepared in Example 4, which reveal two small regions of ruptured atherosclerotic plaque on the wall of the proximal left anterior descending artery. Based on these results the patient is sent to the cardiac cath lab where a stent is placed at the site of plaque instability to structurally support the weakening vascular wall and to preclude progression of luminal thrombus formation or embolization. The patient is placed on aggressive medical therapy and lifestyle modification to promote stabilization of his atherosclerotic disease. and to minimize his potential for future cardiac events.

### EXAMPLE 13

Mrs. C. is a 55-year-old women who presents with symptoms of momentary vision left disturbance and right hand weakness that resolves in less than 4 hours. Carotid duplex ultrasound reveals intact antegrade flow bilaterally with 50% or less diffuse disease. The patient thinks she recalls a similar episode 3 months ago affecting her left hand.

The physician decides to rule-out transient ischemic attacks. The patient undergoes a carotid MR angiogram which confirms good bilateral antegrade flow. Fibrin-targeted nanoparticles as prepared in Example 4 are given to rule ruptured atherosclerotic plaque as an embolic source of transient ischemic attack (TIA). Following administration of the agent, the patient is noted to have multiple, but focal accumulation of contrast identified by t1w MRI imaging in the left common carotid. Based on these findings, a decision is made to surgically remove the plaque through carotid endarterectomy.

1. A method to image and/or treat blood clots in human subjects, which method comprises administering to a subject in need of such diagnosis and/or treatment an emulsion of liquid, high-boiling perfluorocarbon-based nanoparticles, wherein said nanoparticles further comprise a coating of lipid/surfactant and which nanoparticles are coupled directly to at least one targeting ligand that is specific for at least one component characterizing blood clots, and
effecting said imaging or treatment.

2. The method of claim 1, which comprises administering said composition to said human subject systemically.

3. The method of claim 2, wherein said systemic administering is by intravenous administration.

4. The method of claim 1, wherein said method comprises administering said composition locally to said clot.

5. The method of claim 1, which method comprises obtaining an image of said clot using ultrasound, MRI or a radionuclide.

6. The method of claim 1, which method comprises effecting lysis or constrained propagation of said clot.

7. The method of claim 1, wherein said targeting ligand is coupled covalently to a component of the lipid/surfactant coating.

8. The method of claim 1, wherein said ligand binds specifically to fibrin.

9. The method of claim 1, wherein said nanoparticles further include at least one magnetic resonance imaging (MRI) contrast agent.

10. The method of claim 9, wherein said MRI contrast agent is a chelated paramagnetic ion.

11. The method of claim 10, wherein said chelating agent is DOTA and the paramagnetic ion is gadolinium ion.

12. The method of claim 1, wherein said nanoparticles further contain at least one radionuclide.

13. The method of claim 12, wherein said radionuclide is ⁹⁹Tc.

14. The method of claim 1, wherein said emulsion further includes at least one biologically active agent.

15. The method of claim 14, wherein said biologically active agent is a thrombolytic agent.

16. The method of claim 1, wherein said targeting ligand is an antibody, a fragment of an antibody, a peptide, an aptamer, a peptidomimetic or a receptor ligand.

17. The method of claim 16, wherein the targeting ligand is an antibody or fragment of an antibody.

18. The method of claim 17, wherein said antibody or fragment is humanized.
